(19) United States
(12) Patent Application Publication
(10) Pub. No.: US 2014/0296823 A1
(43) Pub. Date: Oct. 2, 2014

(54) Dual-use Catheter for Continuous Analyte Measurement and Drug Delivery

(71) Applicants: The State of Oregon acting by and through the State Board of Higher Education on behalf of Orego, Corvallis, OR (US); Pacific Diabetes Technologies Inc., Portland, OR (US)

(72) Inventors: William Kenneth Ward, Portland, OR (US); Peter G. Jacobs, Portland, OR (US); Robert S. Cargill, Portland, OR (US); Jessica R. Castle, Portland, OR (US); John Conely, Corvallis, OR (US); Gregory Herman, Albany, OR (US)

(73) Assignees: PACIFIC DIABETES TECHNOLOGIES INC., Portland, OR (US); The State of Oregon acting by and through the State Board of Higher Education on behalf of Orego, Corvallis, OR (US)

(21) Appl. No.: 14/304,166

(22) Filed: Jun. 13, 2014

Related U.S. Application Data
(63) Continuation of application No. PCT/US12/69697, filed on Dec. 14, 2012.
(60) Provisional application No. 61/570,382, filed on Dec. 14, 2011.

(51) Int. Cl. A61M 25/00

(52) U.S. Cl. CPC A61M 25/0017; A61M 25/0043; A61M 25/0009; A61M 2025/0057; A61M 2230/005; A61M 2230/201
USPC 604/504; 604/264; 29/428

(57) ABSTRACT

A sensing assembly (10), including a body (12) and one or more first indicating electrodes disposed on the body (26). The first indicating electrodes include an electrochemically active layer (32) and a layer (38) of an active functioning enzyme of a first enzyme type on top of the electrochemically active layer. Also, one or more second indicating electrodes (24) are disposed on the body and include an electrochemically active layer (32) and a layer (36) of an inactivated enzyme of the first enzyme type on top of the electrochemically active layer. A reference electrode (22) is also disposed on the body. Finally, an electrical and data processing system (18) is adapted to bias the electrodes and measure electrical signals from the electrodes, and uses said signals to determine an analyte concentration and communicates the analyte concentration to a location apart from the first and second indicating electrodes.

### RELATED APPLICATIONS

This application is a continuation of application serial number PCT/US12/69697, filed on Dec. 14, 2012 which claims priority from provisional application Ser. No. 61/570,382, filed Dec. 14, 2011 which are hereby incorporated by reference as if fully set forth herein.

### GOVERNMENT SUPPORT

This invention was made with government support under grant number 1R43DK096678-01 awarded by National Institutes of Health. The government has certain rights in the invention.”

### BACKGROUND

The present invention is related to sensing of one or more analyte and delivering one or more drugs in response to the analyte measurements. The present invention is more specifically related to an apparatus for sensing of one or more analyte and delivering one or more drugs in response to the analyte measurements.

Many research groups have developed artificial endocrine pancreas (AP) prototypes, which combine continuous glucose sensing with automated hormone delivery (for example, Weinzimer, S. A., Steil, G. M., Swan, K. L., Dziura, J., Kurtz, N., and Tamborlane, W. V.: Fully automated closed-loop insulin delivery versus semiautomated hybrid control in pediatric patients with type 1 diabetes using an artificial pancreas. Diabetes Care. 2008; 31: 934-9). Some AP technology includes the delivery of two hormones, insulin and glucagon, in response to glucose sensor data and utilizes a model of carbohydrate metabolism (See El Youssef, J., Castle, J. R., Branigan, D. L., Massoud, R. G., Breen, M. E., Jacobs, P. G., Bequette, B. W., and Ward, W. K.: A controlled study of the effectiveness of an adaptive closed-loop algorithm to minimize corticosteroid-induced stress hyperglycemia in type 1 diabetes. J Diabetes Sci Technol. 2011; 5: 1312-1326.) to continually compensate for changes in tissue sensitivity to insulin. In the Castle et al study, it was observed that glucagon plus insulin was much more effective than placebo plus insulin in avoiding overt hypoglycemia. A recent analysis suggests that intensive glucose monitoring with intensive insulin delivery leads to cost savings.

However, there are major limitations to current AP systems. In addition to issues with slow insulin absorption and suboptimal sensor accuracy, current systems are cumbersome. All systems require at least one hormone delivery pump and some require two. All require at least one glucose sensor and some require two. In fact, for optimal safety, many workers believe that the patient should have two subcutaneous glucose sensors and two hormone delivery devices. In addition to the need for multiple body-worn devices (see FIG. 1), the user must carry a sensor receiver(s) and pump controller(s). The multitude of devices makes it difficult to carry out activities of daily living. In addition, bacterial colonization and infection at insertion sites is not unusual; a reduction in the number of insertion sites reduces this risk.

Another problem encountered in the development of an accurate glucose sensor is posed by substances other than glucose that affect the sensor reading. Some compounds such as ascorbic acid, acetaminophen, and uric acid are easily oxidized directly (non-enzymatically) at the surface of a positively polarized indicating electrode such as those made from platinum, carbon, gold or palladium. Some workers in the field have developed membranes (sometimes referred to as “specificity” membranes) that keep these larger molecular weight compounds away from the indicating electrode and at the same time, allow the very small molecular weight analytes such as hydrogen peroxide to diffuse through the membrane (see US patents by Wilson et al, U.S. Pat. No. 5,165,407; and Ward et al, U.S. Pat. No. 6,613,379).

Though such methods are successful to some extent, they also reduce the permeation of the analyte of interest, e.g. hydrogen peroxide. When such membranes are used for permselectivity, it is not possible to completely prevent permeation of the interfering compound while, at the same time, allowing unimpeded permeation of the analyte of interest. Thus, the use of a specificity membrane always reduces the overall sensitivity to glucose, sometimes by up to 90 percent, as compared to a sensor that does not require such a membrane.

Some workers in the field have used a method that avoids the need for a specificity membrane: some electrodes are coated with the enzyme such as glucose oxidase (GOX) and other electrodes are left uncoated. For example, Ward et al in U.S. Pat. No. 6,212,416, B1, teaches creation of a planar sensor electrode array wherein some anodes are coated with enzyme and some are not coated with enzyme. When the signal from the uncoated electrode is subtracted from the signal of the coated electrode, the resulting value is useful and approximately equal to the true glucose concentration, but there is some error. More specifically, such a method is limited in that the path length for the analytes of interest (glucose, interfering compounds, and hydrogen peroxide) is different when one compares the coated vs uncoated electrodes. When the path lengths are different, diffusion characteristics for glucose (coated) electrode and the blank (uncoated) electrode are different. For example, when the path length is longer (in a coated electrode), a greater proportion of the analyte of interest will diffuse back out of the sensor compared to the uncoated electrode and thus lead to a falsely low signal in that electrode. For this reason, this type of simple subtraction system leads to a consistent error.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Herein, we disclose a novel AP technology that consists of a flexible polymer-based catheter into which a continuous amperometric sensor is integrated. This miniaturized device allows more freedom of movement and will increase patient acceptance. Because of its low risk for catheter dislodgement, the subcutaneous location is preferable to the intradermal location, but the device can also be used in the intravascular location.

Disclosed here are methods for creating an analyte sensor (or sensor array), for example a glucose sensor, disposed on the outer surface of a tube. Together, the sensor and catheter comprise a single unified device with no need for a second catheter, needle, or distant electrode. This device is used both to continuously measure the concentration of an analyte such as glucose in blood or in tissue and to serve as a conduit through which drugs such as insulin and/or glucagon can be delivered. Due to the configuration of a novel “subtraction system” in which the current of the inactive enzyme electrodes is subtracted from the current of the active enzyme electrodes to yield the true analyte signal, there is no need for a separate specificity membrane to prevent oxidizable interferents from reaching the indicating electrode. The lack of the need for a specificity membrane favors simple manufacturability of this device and minimizes loss of analyte signal strength.

In some embodiments, the lumen is filled with porous material to decrease the size of the dead volume. When two or more drugs are being delivered by a single catheter, a large dead volume creates a large drug delivery error when delivery of drug 1 is stopped and delivery of drug 2 is started.

One embodiment is not a microneedle or microcatheter (both of which are defined as having a length of 1 mm or less). Instead, it is a macrocatheter, defined as having a length of 5 mm or greater. Advantages of a macrocatheter include larger electrode area for each indicating (working) electrode and the capability to place many indicating electrodes on the surface of a single catheter.

The indicating electrode and combined reference and counter electrode are disposed on the same catheter, obviating the need for a separate catheter or needle to serve as a second electrode.

This unified device is created by microfabrication techniques used in the semiconductor industry, including deposition, photolithography, and etching, as well as direct additive fabrication using printing techniques. This device can deliver insulin, glucagon, pramlintide, and/or others. A version of this device is disclosed in which there are multiple sensing units, each of which is separated from the others. In such an embodiment with multiple sensing units, the accuracy of the sensing system is increased due to the concept of redundancy.

In one embodiment, the starting material is a flexible polymer substrate in the form of a sheet or a web, which will be termed a flat surface, (for example, polyimide, though other materials can be used) on which metals, dielectrics, and materials with specific functionality are deposited and patterned to create the capability to measure glucose continuously. The flexible polymer substrate is then wrapped into a tube or wrapped around and cemented to a preexisting tube so that the tube can be used as a conduit. The conductive traces from this sensing catheter are routed into a miniaturized electrical current amplifying circuit which acquires the analyte current and then provides an analyte (e.g. glucose) value. This device can serve as an artificial pancreas when the sensed glucose value is ported to an algorithm that automatically determines the correct insulin and/or glucagon rate to keep the glucose well controlled. Based on commands from the algorithm, the hormones are pumped through the lumen of the tube into the subcutaneous tissue or the blood of the patient with diabetes.

In another embodiment, the starting material is not a flexible polymer substrate in the form of a sheet or a web but a polymeric tube. Metals, dielectrics and materials with specific functionality are deposited and patterned to create the capability to measure glucose continuously. Due to the radius of curvature of the polymeric tubes, printing methods are desirable to pattern the materials. Methods suitable for printing directly on the surface of the tube include microcontact, embossing, or non-contact printing directly on the surface of the tube. These printing methods can be used to print resists or self assembled monolayers, but also to print metals, dielectrics, electrode compounds, GOX, and other functional materials. Due to the small radius of curvature the use of a very precise printing method, electrohydrodynamic printing (EHDP), in which the drop sizes are extremely small, allows ultra-high resolution printing on to a curved surface without spreading the materials beyond the intended locations.

In the flat substrate method or the pre-formed tube method, the enzyme layer is applied with a simple 3 step printing method, disclosed below, that avoids the need for a separate specificity membrane.

This sensing system can be integrated into the outer surface of a flexible tube and manufactured at low cost. The geometry used in wire-based sensor, i.e. a solid rod, cannot be used for drug delivery. Insulin and glucagon could be delivered from separate reservoirs through the single catheter of this device.

Despite efforts in manufacturing process development, it has been very difficult to achieve consistency in sensor function—reports emphasizing the sensor-to-sensor variability have been published. A microfabricated system will have very low sensor-to-sensor variability.

It should be noted that one previously disclosed method for the creation of a single port sensing and drug delivery catheter requires fluid to be perfused continuously or intermittently through the catheter, such as the method of microdialysis (see patent Ser. No. 11/910,096-2009/0005724). In contrast, the method disclosed here includes creating a sensing catheter without the need for fluid perfusion. Herein, we also disclose a method for starting with a tube, (radiused surface) and using a highly-precise printer with ultra small drop size, applying the electrode layers, enzyme layer, and outer permselective layers with this printer. This alternative method has the advantage of being a single tube with no seam, and thus avoids the potential issue of rupture or leaks.

To avoid measurement error caused by interference substances, we have developed a method that uses active glucose oxidase (GOX) and inactive GOX (GOX which has been thermally or chemically denatured). This method overcomes this disparity in path lengths by making the path lengths identical and by making the protein matrix through which the analytes diffuse identical (except that in one case, the protein enzyme is functional with proper tertiary folding, and in the other case, non-functional). Using such a method, the interfering current (current obtained from inactive enzyme electrode(s)) is subtracted in real time from the total current (glucose plus interfering compound current obtained from the active electrode). The result yields the current due exclusively to glucose without the need for an additional specificity membrane.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is an illustration of prior art sensors and drug delivery units in place on a human abdomen.

FIG. 2a is a side perspective view of a preferred embodiment of a sensing macrocatheter assembly section, according to the present invention.

FIG. 2b is a side perspective view of an entire sensing macrocatheter assembly, shown threaded on an introducer.

FIG. 3a shows a top view of a completed set of electrodes, for placement on the macrocatheter of FIG. 2a.

FIG. 3b shows a mask for deposit of an electrochemically active, conductive layer for the electrodes of FIG. 3a.

FIG. 3c shows a mask for deposit of an insulating material, for the electrodes of FIG. 3a.

FIG. 3d shows a mask for further deposit of conductive material, to form contacts, for the electrodes of FIG. 3a.

FIG. 4a is a sectional view of the electrodes of FIG. 3a, but missing a permselective layer.

FIG. 4b is a sectional view of the electrodes of FIG. 3a in an alternative embodiment where the gold layer is eliminated, and along a different view line than 4a, also showing only the active GOX electrode.

FIG. 5 is a top view of a sheet of electrodes, to be sectioned for placement on a macrocatheter tube.

FIG. 6 shows a sensing macrocatheter assembly, having multiple active sensor pairs.

FIG. 7 shows a sheet of sensors, to be sectioned.

FIG. 8 shows micrographs of porous material filling catheter tube, with the left side micrograph showing porous material having a porous material density of 6% and the right side micrograph showing porous material having a porous material density of 65%.

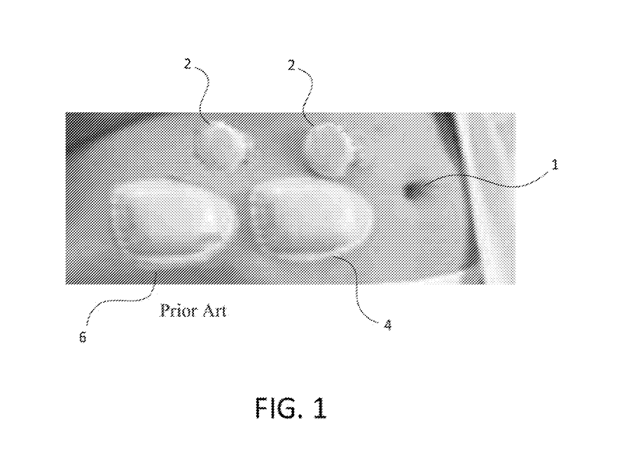

### BEST MODES OF CARRYING OUT THE INVENTION

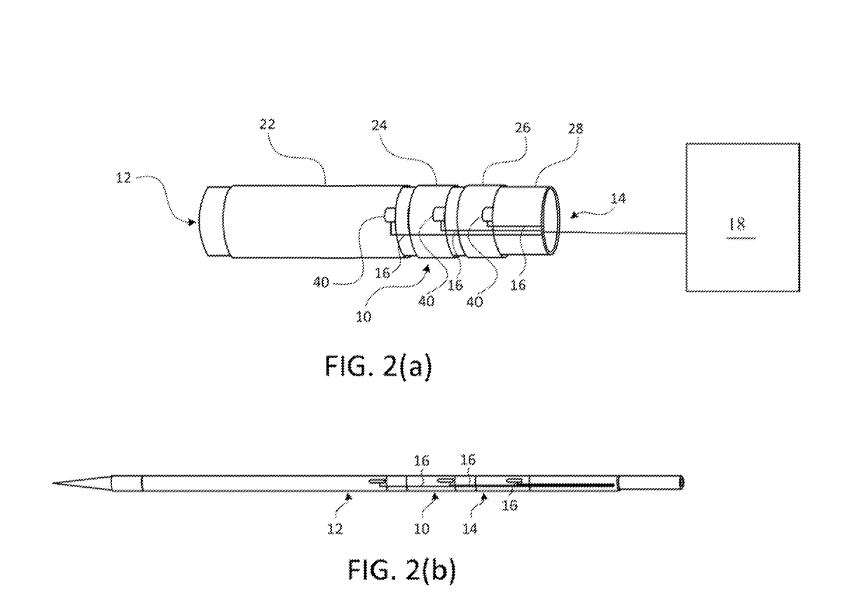

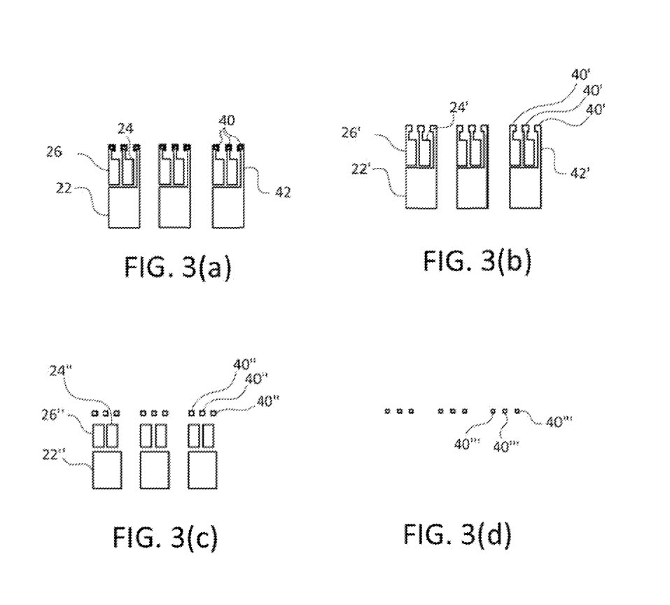

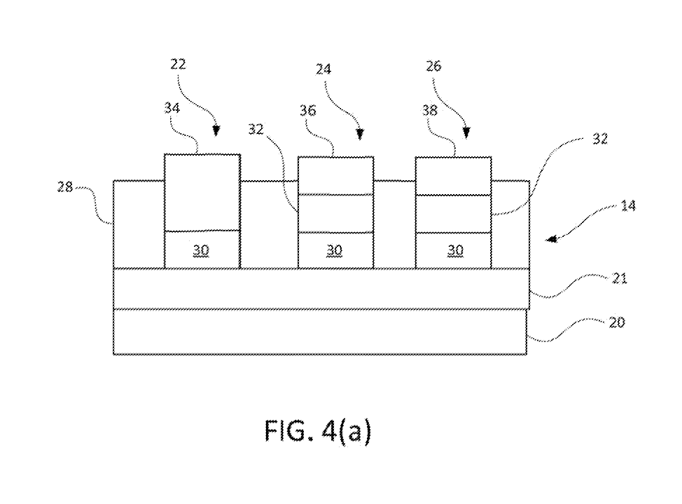

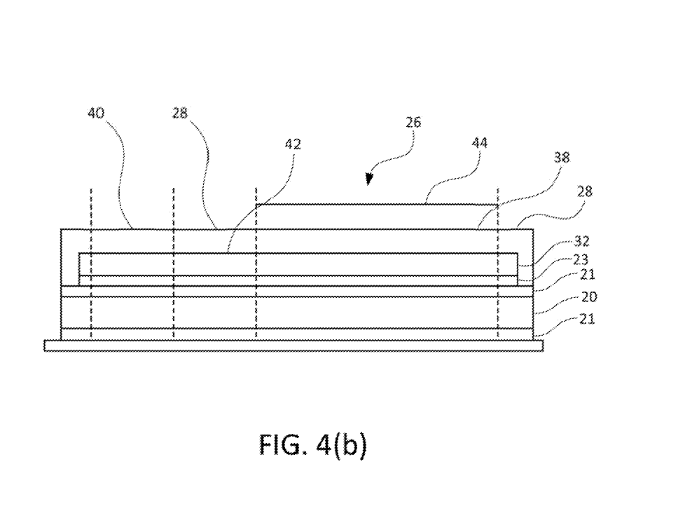

Referring to FIGS. 2 and 3, a sensing catheter assembly 10 includes a catheter 12 in the form of a round tube and having an exterior surface that supports a sensor assembly 14. A set of conductive traces 16 connect sensor assembly 14 to an electrical and data analysis system or assembly 18. Sensor assembly 14 includes a flexible polyimide base 20 that is adhered to catheter 12. An SiO₂ layer 21 is fixed to base 20, and in turn supports three sensing electrodes: an Ag/AgCl reference electrode 22; an inactive GOX indicating electrode 24 and an active GOX indicating electrode 26. A second layer of polyimde 28 surrounds each electrode 22, 24 and 26, with only the top portion of elements 22, 24 and 26 protruding from layer 28. Each electrode 22, 24 and 26 includes a base of gold 30 adhered to layer 21 with a thin layer 23 of an adhesion promoter such as titanium, nickel, tantalum or chromium (FIG. 4[b]), and indicating electrodes 24 and 26 each include a layer of platinum 32 over the gold base 30. Finally, electrode 22 has a layer of Ag/AgCl 34 directly on gold base 30, electrode 24 has an inactive layer of GOX 36, on top of platinum layer 32, and electrode 26 has an active layer of GOX 38, also on top of platinum layer 32. Traces lead to connector tabs 40.

### Microfabrication Methods

Creation of the integrated sensor and infusion catheter is carried out in two stages: (1) microfabrication of the sensing units in a flat configuration, which involves insulator deposition, metal deposition, specialized ink printing, enzyme deposition, and permselective polymer coating; and (2) wrapping the sheets into units which are individualized into sensing catheters; for measuring sensor signals, it is simple to use standard electronic devices to measure and record sensor data on the bench and in vivo.

Referring to FIGS. 2, 3 and 5, a polyimide substrate 20 (which may be obtained from American Durafilm, Inc., having a website address of “www.americandurafilm.com”), is nominally 50 μm thick, though this thickness is not crucial. Prior to processing the polyimide materials will be degassed in a vacuum oven up to the maximum process temperature to be used during the fabrication process. The surface is treated with an Ar or O₂ plasma prior to the application by atomic layer deposition of an inorganic insulator layer 21 which may include Al₂O₃, SiO₂, Si₃N₄ or other compounds. Insulator layer 21 minimizes the uptake of moisture of solvents during further processing, and also improves adhesion of the next deposited metal layer (see below).

Good adhesion is crucial to help prevent fracture at the metal/substrate junction during wire bonding and the subsequent wrapping of the substrate around the cylindrical tube. Gold (layer 30) and titanium or chromium 23 are deposited on the surface of the Al₂O₃ layer 20, where the titanium 23 is an adhesion layer and is deposited during the same process and is located between the gold layer 32 and Al₂O₃ layer 20. These metal layers (layer 30, titanium adhesive layer) also serve as the conductive traces from the electrode to the connector tabs 40 on the sensor edge. Positive photoresist is applied to the metal surface and soft baked. A diagram of the sensing catheter is shown in FIG. 2 and the mask configuration is shown in FIGS. 3(b)-3(d). FIG. 3(a) shows a completed electrode assembly 14 or portion thereof, having indicating electrodes 24 and 26, reference electrode 22, traces 42 and contacts 40. The mask in FIG. 3(b), shows locations of active GOX electrode 26′, the inactive GOX electrode 24′ the reference electrode 22′, the tabs 40′ and the traces 42′. This mask is used during UV exposure and the resist is developed and hard baked. The gold and titanium layers will then be etched away everywhere except under the resist (unremoved due to the mask) thereby thereby creating the sites for the indicating electrodes. Platinum iridium alloys are the material for the indicating electrodes 24 and 26 and they are applied by physical vapor deposition (sputter deposition, evaporation, etc), and patterned using similar fabrication processes as described above. An alternative to the vapor phase deposition of platinum iridium alloys is the printing and/or plating of these materials using solution based processing. This can occur through the direct printing using nanoparticulate platinum iridium inks, the printing molecular precursors of these species, or the electrolytic or electrolysis plating of platinum iridium films. For deposition of the reference electrode (Ag/AgCl), the preferred method is using a Ag/AgCl ink (available from either Ercon: www.ercon.com, or Novacentrix, www.novacentrix.com), applied by micro-scale printing. Nanoparticle silver ink or silver precursor inks are stable and do not oxidize, though under some conditions heat-curing is necessary but may distort the polyimide substrate. An alternative method to form the reference electrode is physical vapor deposition of Ag and etching using the mask shown in FIG. 3(c), showing electrode locations 22″, 24″ and 26″ and contact locations 40″. FIG. 3(d) shows mask used for final steel deposition for contacts 40′.

A polyimide insulating layer is then applied over all layers and etched so that only the connector tabs 40 and the electrodes are uncoated. Active GOX 38 is then applied to one of the sensor sites and inactive GOX 36 is applied to the other as shown in FIG. 4(a), which shows a cross section of all the layers. FIG. 4(b) shows an embodiment in which platinum is used in place of gold layer 32, thereby simplifying the fabrication process.

There are many methods that are suitable to inactivate or denature glucose oxidase (GOX). It is simplest to denature the GOX before it is applied on to the surface of the electrode, though it can be inactivated after application. One chemical method for denaturation is the addition of guanidinium HCl (GdmCl) prior to deposition on to the indicating electrode. It was shown that the addition of GdmCl in a concentration of 6-8 M was sufficient to completely denature GOX (Akhtar, M. S., Ahmad, A., and Bhakuni, V.: Guanidinium chloride- and urea-induced unfolding of the dimeric enzyme glucose oxidase. Biochemistry. 2002; 41: 3819-27). Urea (6-8 M) can also be used for this purpose. Some surfactants can be used to denature GOX, such as dodecyl trimethyl ammonium bromide (DTAB), ionic detergents such as sodium dodecyl sulfate (SDS) and hexadecyltrimethylammonium bromide as shown by Housaindokht (Housaindokht, M. R. and Moosavi-Movahedi, A. A.: Determination of binding affinities of glucose oxidase for sodium n-dodecyl sulfate. Int J Biol Macromol. 1994; 16: 77-80). Another method to denature GOX is the use of micromolar amounts of heavy metals such as mercury, lead and silver. Other denaturation methods include the use of proteases or the use of ultraviolet irradiation.

Another method to inactivate GOX is to remove the cofactor (flavin adenine dinucleotide, FAD) that allows GOX to alternate between oxidized and reduced forms. Without the FAD, GOX is completely inactive. The FAD cofactor for GOX can be removed by sulfuric acid, as described by Ngo (Ngo, T. T. and Lenhoff, H. M.: Antibody-induced conformational restriction as basis for new separation free enzyme immunoassay. Biochem Biophys Res Commun. 1983; 114:1097-103). The resulting inactive compound is the GOX apoenzyme without the prosthetic cofactor. Because the cofactor constitutes such a tiny fraction of the holoenzyme, the diffusion characteristics through APO-GOX are identical to diffusion characteristics of the holoenzyme, HOLO-GOX. To be absolutely certain that the inactive enzyme is devoid of all activity, one might wish to combine several of these methods, such a chemical denaturing method and a method to remove the FAD cofactor.

Application of heat (above 50-70 degrees C.) is also a well know method for inactivation of glucose oxidase. However, thermal denaturation is usually accompanied by some protein aggregation or gelation, that is, forming microscopic or macroscopic polymers or gels. Some of these gels are small and remain soluble but application of prolonged heat can lead to large, insoluble gels. Because aggregation can change the nature of diffusion through the enzyme, the preferred means for inactivating GOX is to use chemical methods. For the coating of the inactive electrode, one can also use proteins other than the enzyme used for the active electrode.

### Application of GOX to Electrodes

There are numerous methods by which GOX can be applied to indicating electrodes. It can be pipetted, printed, or applied by a silk screen process, as reviewed by Honeychurch (Honeychurch, K. C.: Screen-printed Electrochemical Sensors and Biosensors for Monitoring Metal Pollutants. Insciences J. 2012; 2: 1-51). Several workers showed that piezoelectric inkjet printing can be used to deposit GOX, including Yun {Yun, 2011 #36} and Cook/Derby {Cook, 2010 #37}. Setti et al showed that despite heating the enzyme, thermal inkjet printing can be used to deposit GOX with minimal loss of enzyme activity (Setti, L., Fraleoni-Morgera, A., Ballarin, B., Filippini, A., Frascaro, D., and Piana, C.: An amperometric glucose biosensor prototype fabricated by thermal inkjet printing. Biosens Bioelectron. 2005; 20: 2019-26). Gonzalez-Macia has reviewed many methods of depositing glucose oxidase by printing methods (Gonzalez-Macia, L., Morrin, A., Smyth, M. R., and Killard, A. J.: Advanced printing and deposition methodologies for the fabrication of biosensors and biodevices. Analyst. 2010; 135: 845-67). In some cases, very small feature size for printed materials are needed, for example, when one needs a large number of electrodes in a small area. In such a case, when extremely fine (high resolution) enzyme printing is needed, electrohydrodynamic printing (EHDP) is well-suited, as disclosed by Rogers et al (applications Ser. No. 12/916,934-US 2012/0105528 A1 and Ser. No. 12/669,287US and 2011/0187798 A1. This EHDP method avoids high heat and can print down to one micron feature size.

It is critical to avoid loss of GOX into the tissue of the patient who is using the sensor. For this reason, the GOX must be immobilized so that it cannot leach out. A convenient method of crosslinking GOX so that it remains tightly bound to the substrate and the carrier protein is to use glutaraldehyde, for example in the concentrations discussed in House et al (House, J. L., Anderson, E. M., and Ward, W. K.: Immobilization techniques to avoid enzyme loss from oxidase-based biosensors: a one-year study. J Diabetes Sci Technol. 2007; 1: 18-27). It is important to note that glutaraldehyde by itself is stable in liquid solution, but when added to a protein solution, it will quickly form chemical bonds, especially with the amine groups, but also with thiol, phenol, and imidazole groups. For this reason, the optimal method disclosed here is to print the GOX enzyme and crosslinker in two steps and allow the crosslinking to occur after printing. If the glutaraldehyde is mixed first with the GOX solution, it must be printed immediately after mixing, and the lines and nozzles rinsed completely and immediately to avoid crosslinking and damaging the equipment.

Three-Step Method of Printing GOX on to Electrodes—with the Following Ratios: GOX, Approx. 27,000 Units per ml; Albumin, Approx. 1.75 weight %; and Glutaraldehyde 0.6 Weight %.

Note that these amounts disclosed below can be scaled up or down, as needed. Gelatin or other proteins can be used in the place of albumin. Before this printing procedure is carried out, the metallized electrodes must be deposited as described elsewhere in this document.
STEP 1: Preparation of active GOX, water, protein extender (volume 36.9 ml)
   1. Obtain GOX, 200-300 units per mg, usual strength is approx. 285 units per mg.
   2. Add 3725 mg of GOX to water to bring total volume to 21.3 ml at neutral pH.
   3. Add 775 mg of bovine serum albumin or human serum albumin to purified water so that the albumin solution volume is 15.5 ml.
   4. Gently mix the GOX solution with the albumin solution, do not shake. Final volume: 36.9 mL
   5. Print and pattern this solution on to the electrodes.
STEP 2: Preparation of inactive or denatured GOX, water, protein extender (volume 36.9 ml)
   1. Pretreat GOX with GdmCl (6-8 M), urea (6-8 M), or remove the prosthetic FAD cofactor with sulfuric acid according to the method of Morris (Morris, D. L. and Buckler, R. T., Glucose Oxidase, in Methods in Enzymology, H.V.V.E. in: J. J. Langone, Editor. 1983. p. pp. 413-425). To guarantee complete absence of any GOX activity, the GOX can be treated with GdmCl or urea AND treated with sulfuric acid.
   2. Add 3725 mg of inactivated, purified GOX to water to bring total to 21.3 ml of solution at neutral pH.
   3. Add 775 mg of bovine serum albumin or human serum albumin to purified water so that the total albumin solution volume is 15.5 ml.
   4. Gently mix the GOX solution with the albumin solution, do no shake. Final volume: 36.9 ml.
   5. Print and pattern this solution on to “inactive” electrodes, not the same electrodes as for the active GOX.
STEP 3: Preparation and application of glutaraldehyde and water; (final volume: 3.1 ml)
   1. Obtain solution of 25% glutaraldehyde in water.
   2. Dilute this solution with purified water so that the concentration of glutaraldehyde becomes 12.5%
   3. Measure out 3.1 ml of this 12.5% solution.
   4. Print and pattern this solution on to both types of electrodes (active and inactive GOX).
   5. Allow this enzyme+glutaraldehyde mixture to cure (cross link) for at least 2 hr at 40 deg C. before handling the substrate. Immerse the electrodes in purified water or phosphate-buffered saline for 24 hours to wash off unreacted enzyme, albumin and glutaraldehyde.

An alternative method is to print active GOX on both the “active” and “inactive” indicating electrodes, followed by a step in which the GOX is thermally deactivated on the inactive electrodes. This can be accomplished by precise heating using an infrared laser or a microfabricated resistor. The microfabricated resistor can also be used to monitor the temperature of the sensor system.

As alternatives to glutaraldehyde, other crosslinking agents such as sucidyl suberate and carbodiimide can be used.

After the active and inactive GOX coatings have been prepared and cured, it is necessary to deposit an outer membrane that limits diffusion of glucose and allows permeation of oxygen so that the dynamic response to glucose occurs over a large range of glucose concentration. There are many possibilities for the materials for such permselective membranes, which include silicone compounds (polydimethyl-siloxane), polyurethanes, Nafion, sulfonated poly (ether, ether, ketone), polyester sulfonic acids, and hydrogels. Typically, there is a need for the material to have hydrophilic moieties and hydrophobic moieties to regulate permeation of hydrophilic analytes such as glucose and hydrophobic moieties such as oxygen. Acrylates can be used as the permselective membranes and have the advantage of being curable by ultraviolet irradation. Direct patterning techniques, such as ink jet, transfer, offset, Gravure, or other printing methods, are suitable for deposition and patterning of the permselective layer.

### The Benefit of a “Macro-Catheter” and the Benefit of Multiple Sensing Units.

The disclosure of Gonnelli (US 3009/0062752 A1) cites the benefits of a microneedle (such as reduced pain), defined as less than 1 mm in length. One catheter assembly embodiment is distinguished from Gonnelli in that a “macro-catheter” is preferred. For a high signal to noise ratio (SNR), adequate signal strength is crucial. In particular, there is a need for sufficiently large electrode area, primarily of the indicating electrode (working electrode). Noise is a problem, for example from physical jarring of a sensor or electrical noise from environmental equipment. A larger signal from the analyte of interest minimizes noise-induced inaccuracy. Mathematical smoothing can reduce noise, but induces a delay, which is very harmful to a closed loop system accuracy.

There are two situations in which there is a very substantial benefit of a macro-catheter (whose length of at least 5 mm) which is used both for sensing and drug delivery. The first situation is when there is a need for two different types of indicating electrodes, one with active enzyme (e.g. glucose oxidase) and one with inactive enzyme, as discussed above. The benefit of using both inactive and active enzyme is that the active enzyme measures glucose and other interfering compounds such as ascorbic acid, acetaminophen and uric acid; whereas the electrode coated with inactive enzyme only measures the interfering compounds. In a microneedle or microcatheter, there is less area available on the catheter for the two electrodes and therefore, the signal strength will be lower in a microneedle, all other things being equal.

The other situation favored by a macro-catheter is the need for multiple sensing units distributed over a large area. The present applicants have shown in two previous studies that the use of multiple sensing units leads to better sensor accuracy. As the number of active indicating electrodes rises, there is a need for more electrode surface area to maximize signal strength, favoring the need for a macro-catheter whose length is at least 5 mm.

### Addressing the Problem of Dead Volume in a Bihormonal Delivery Catheter

To minimize the number of devices needed in a bihormonal artificial endocrine pancreas, it is desirable to use a single subcutaneous catheter for the infusion of both insulin and glucagon. In addition, in one catheter assembly embodiment, we also describe the use of this catheter to serve as a continuous electrochemical glucose monitor (amperometric or coulometric sensor). On can term this embodiment a triple use catheter assembly because it measures glucose continuously, and it serves as a delivery conduit both for insulin and glucagon. However for such a combination device to function successfully, there are several requirements that are not typical of standard medical catheters. Specifically, the internal dead volume must be minimized for optimal function of such a catheter.

The internal dead volume is the capacity of liquid within the catheter. It is termed “dead” because a drug residing in it has not yet reached the tissue of the patient and is thus not available. It is important to understand the concept of the “available liquid fraction” (ALF). The ALF is the “available liquid volume” in the interior of a catheter divided by the “total internal volume” of that catheter. As an example, let us consider a catheter whose internal dimensions are 0.5 mm in diameter and 10 mm in length. The total interior dead volume is 2 cu mm. If this volume is filled with U100 insulin (defined as 100 units per ml), this volume holds 0.2 units. If it is filled with U500 insulin (500 units per ml), the volume holds 1 unit of insulin. If the system has been delivering U500 insulin and now suddenly changes to the delivery of glucagon (because glucose level is falling), the dead volume of insulin (1 unit) will be suddenly delivered, i.e., it will be pushed into the patient's tissue ahead of the glucagon bolus. To many persons with type 1 diabetes, the sudden infusion of 1 unit of insulin can be quite detrimental and can further exacerbate the tendency toward hypoglycemia. The situation is not so harmful with U100 insulin; however there is an advantage of U500 insulin, as discussed below (see the issue of tissue glucose dilution).

If, on the other hand, there is a foam material within the lumen of the catheter that prevents the dead space from being completely filled with insulin, the overdelivery of insulin is partially ameliorated. As shown in row 3 of Table 1, let us imagine a foam material with interconnected caverns that takes up 50% of the available internal dead volume. In such a case, the actual dead volume is 1 cu mm rather an 2 cu mm which would have been the case if there was no foam. In such an example, there would have been only 0.1 unit of U100 insulin or 0.5 units of U500 insulin delivered when it is necessary to delivery glucagon. The upper panel of Table 1 delineates the incorrect delivery of insulin (when pushed in by another drug such as glucagon) in the case of two different lengths of catheters whose internal diameter is 0.5 mm. It can be seen that for the longer catheter (15 mm) when U500 insulin is indwelled within the catheter, there is a very substantial overdelivery of insulin (1.5 units) when a second drug pushes the insulin into the patient's tissue. The lower panel of Table 1 shows similar calculations for overdelivery of glucagon (at two glucagon concentrations, 1 mg/ml and 2 mg/ml). In the case of glucagon at 2 mg/ml, there is an overdelivery of almost 6 μg glucagon when insulin pushes the glucagon from the dead space into the patient in the situation in which there is no foam to reduce the actual dead volume.

TABLE 1
dead volume: (available liquid volume)/(total internal volume)

| | | Dimensions of catheter | | | Dimensions of catheter | | |
|---|---|---|---|---|---|---|---|
| | | 0.5 mm × 10 mm | | | 0.5 mm × 15 mm | | |
| | INSULIN | | | | | | |
| | | | insulin | insulin | | insulin | insulin |
| porous | | dead | units in | units in | dead | units in | units in |
| material | available | volume | dead | dead | volume | dead | dead |
| density | liquid | (cubic | volume | volume | (cubic | volume | volume |
| fraction | fraction | mm) | U100 | U500 | mm) | U100 | U500 |
| 0 | 1 | 2.0 | 0.20 | 1.0 | 2.9 | 0.29 | 1.5 |
| 0.25 | 0.75 | 1.5 | 0.15 | 0.7 | 2.2 | 0.22 | 1.1 |
| 0.50 | 0.5 | 1.0 | 0.10 | 0.5 | 1.4 | 0.14 | 0.7 |
| 0.75 | 0.25 | 0.5 | 0.05 | 0.2 | 0.7 | 0.07 | 0.4 |
| | GLUCAGON | | | | | | |
| | | | glucagon | glucagon | | glucagon | glucagon |
| porous | | dead | μg in | μg in | dead | μg in | μg in |
| material | available | volume | dead | dead | volume | dead | dead |
| density | liquid | (cubic | volume | volume | (cubic | volume | volume |
| fraction | fraction | mm) | 1 mg/ml | 2 mg/ml | mm) | 1 mg/ml | 2 mg/ml |
| 0 | 1 | 2.0 | 1.96 | 3.9 | 2.9 | 2.95 | 5.9 |
| 0.25 | 0.75 | 1.5 | 1.47 | 2.9 | 8.7 | 2.21 | 4.4 |
| 0.50 | 0.5 | 1.0 | 0.98 | 2.0 | 5.8 | 1.47 | 2.9 |
| 0.75 | 0.25 | 0.5 | 0.49 | 1.0 | 2.9 | 0.74 | 1.5 |

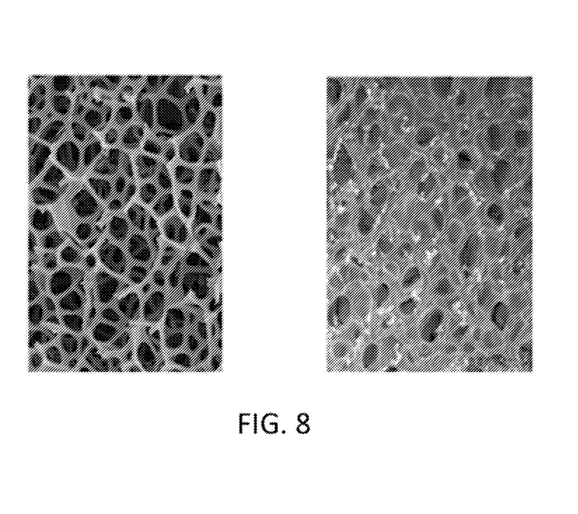

Many types of foam or sponge material (or other porous material) can be used to reduce the actual dead volume. The material can be added during the wrapping of the sensing catheter or can be added after the catheter has already been formed. Examples of materials that can be used to create a porous or sponge material include expanded polytetrafluoroethylene, polyvinyl alcohol, silk, collagen, cellulose, poly-l-lactic acid, chitosan, and materials made porous by salt leaching. Pores or holes can be created in many polymers by the use of rapid solvent evaporation (e.g. by the use of a high vapor pressure solvent such as tetrahydrofuran). In one catheter assembly embodiment, some of the pores must be connected so that fluid infused in the proximal end of the catheter will be forced out the other end instead of being trapped. For diagrams of sponge or foam material with differing porous material density fractions, see FIG. 8.

The above-described catheter assembly embodiment can be distinguished from the sensing catheter disclosed in patent application Ser. No. 11/382,674 (Ward et al), for at least the reason that this reference does not disclose a catheter having a foam-sponge interior.

The Problem with a Single Small Bore Lumen and the Need for Catheter Flexibility.

Flexible catheters have a risk for kinking, a common problem in insulin pump users. Kinks can block delivery of insulin and must be carefully avoided. Repeated kinks can weaken the catheter wall and can break circuits in metallized electrode structures. One advantage of filling the lumen of the sensing catheter with a porous sponge is that the rigidity provided markedly decreases the risk that the catheter will become kinked, or otherwise deformed. Although a sensing catheter could be made with a thick wall (which would create a single small lumen), such an approach is risky because a single, small bore lumen creates a greater risk for occlusions such as that which could occur at the distal tip in the case of a platelet clot or fibrin clot. A foam or sponge material that has many distal openings has less risk for occlusion.

### Avoiding the Need (1) for a Second Catheter and (2) for Intermittent Polarization

As distinguished from the disclosure of Gross (U.S. Pat. No. 5,820,622), the present disclosure consists of a sensing catheter comprising a single structure. This structure does not have a second catheter or needle, or distant electrode, any of which add complexity and discomfort to the patient during insertion. In addition, the Gross disclosure requires a periodic or intermittent polarization potential, in contrast to the present disclosure which uses a continuous, fixed polarization. Although there is one benefit of intermittent polarization (large current), there is a major problem: The current never reaches equilibrium; thus, the analyte signal is very difficult to measure. With intermittent polarization, the current declines sharply immediately after the potential is applied, for two reasons (1) there is a buildup of hydrogen peroxide when the potential is off; and (2) every time the potential is applied, the platinum (or other indicating electrode surface compound) undergoes oxidation. Both of these problems are avoided when the polarization is applied continuously and when the first measurement is not taken until several hours after starting polarization.

### The Issue of Tissue Glucose Dilution, the Issue of Pump Capacity, and the Resulting Need for Concentrated Insulin (Which Leads to a Need for Very Low Dead Volume).

When a catheter that combines analyte sensing in subcutaneous interstitial fluid (ISF) with delivery of hormones or other drugs, the drugs will dilute the ISF before the drug can be absorbed. When drug delivery is high, there is more dilution (falsely low values of glucose or other analyte) and when the drug delivery is off, there is less dilution. To minimize this dilution artifact, the use of a highly concentrated insulin is desirable. Most insulin available today is U100 (100 units per ml) but more concentrated insulins are also available, such as U500 R insulin available from Lilly. The ISF dilution error with U500 insulin would be only 20% of what it would be from standard U100 insulin. But, as mentioned above, the use of U500 insulin can create a problem if there is a large internal catheter dead volume; hence the need for foam or sponge within the catheter.

Other Sensor Design Details: The design of the Ag/AgCl reference+counter electrode requires that its area be large relative to the indicating electrode. A large reference electrode minimizes the probability of significant loss of AgCl over the one week use period {Shinwari, 2010 #4}. There are two methods which can be used to create the reference electrode: ink jet printing or sputtering of silver in a thin film followed by electrolytic chloridization or chloridization with ferric chloride to create Ag/AgCl electrodes.

There are also several methods which can be used for depositing the indicating electrodes, one of which is physical vapor deposition (PVD), sputtering or evaporation of platinum iridium alloys (Pt). Alternative elements to oxidize H₂O₂ include platinum, gold and palladium. Though standard microfabrication processes leads to poor utilization of the patterned materials, it yields high purity and control over the properties of the films. The alternative is using a platinum iridium ink (e.g. obtained from Ercon or other vendor) that can be applied by printing, or by direct plating of the platinum iridium film. After creation of the sensor, the indicating electrodes are continuously polarized between 500 and 650 mV.

The glucose oxidase layer (GOX) is immobilized on the indicating electrodes. GOX may be allergenic during human tissue exposure. Glutaraldehyde (GLUT) concentrations that are too low lead to enzyme loss due to reduced crosslinking. GLUT concentrations that are too high led to reduced glucose sensitivity, due to steric hindrance; without an albumin extender, the enzyme layer becomes brittle and flaky, exacerbated by the wrapping process with flexion. For specific ratios of GOX, glutaraldehyde, and albumin, see paragraph entitled “Three-step method of printing GOX on to electrodes.”

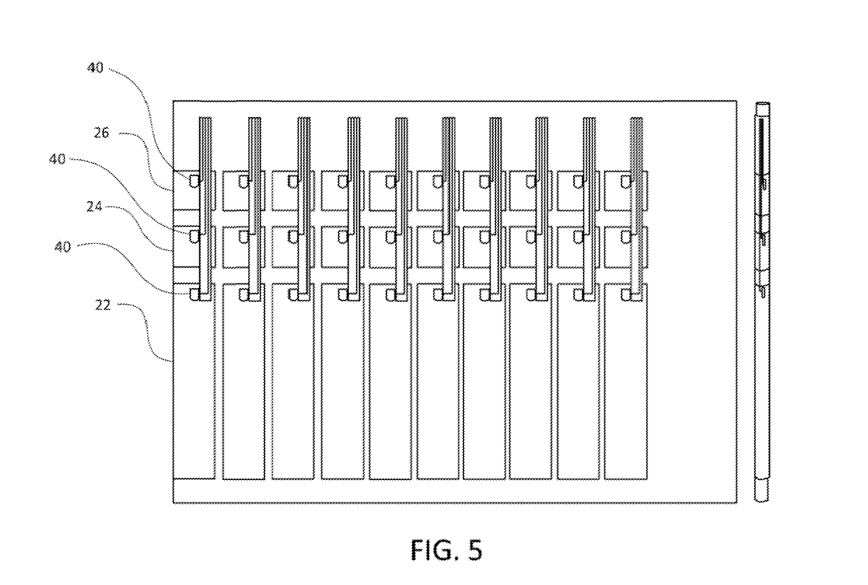

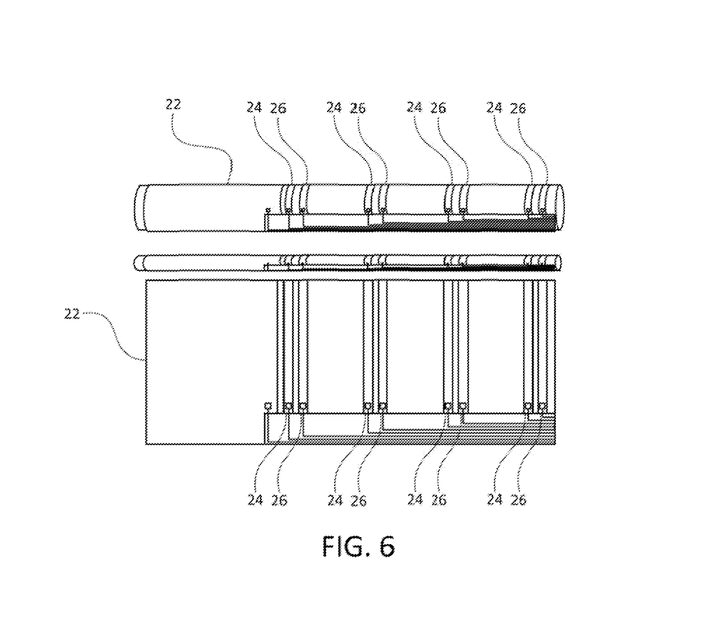

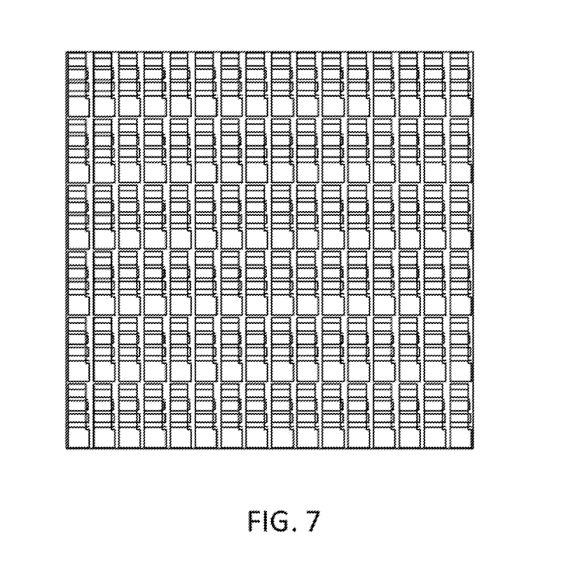

Individualizing the units and creating the catheter: The completed sheets have the layout as shown in FIG. 5. The individualization of the sensors can be carried out by one of two processes. In the first, the sheet of sensors are cut into units after wrapping the units around a separate tube, as discussed below. There is a sacrificial free edge at one end of the substrate for grasping, in order to keep the sheet tight during the wrapping process. The layered polyimide substrate will not be cut into individual rectangles before wrapping. Individualization is carried out after wrapping and bonding. The preferred method for wrapping the sensor substrate into a tube is as follows;
1. Custom-made polyimide tubes are obtained (for example, Microlumen Medical tubing, Tampa Fla. or other vendors) with an OD of 550 μm, an ID of 450 μm, and a wall thickness of 50±5 μm. A mandrel is placed into the tube.
2. Mayer of biocompatible cyanoacrylate (Dymax) cement or other biocompatible cement is microdispensed on the outer surface of the tube, including the leading edge of the sensor unit. Additional lines of cement are dispensed at intervals along the circumference of the tube. The leading edge of the sensing sheet is first cemented to the tube. Micrograspers are used to hold the sacrificial area of the sensing sheet to assist in tight wrapping. After curing, the mandrel is rotated in order to carry out the wrapping (and cementing) process.
3. A knife- or laser cut is made to individualize the sensor.

A custom built machine is used to grasp the sacrificial edge of the polyimide sheet and to carry out the wrapping/individualization process.

Design of a Sensing Catheter with Multiple Sensing Units (Each of Which is Individually Addressable)

With current sensor designs, there is major sensor inaccuracy due to biofouling and drift of unknown origin. In addition, there are substantial manufacturing issues (poor repeatability of manufactured sensors—each one is different from the others). In addition, for an artificial endocrine pancreas, the sensor must be integrated with the insulin pump (and glucagon pump).

This current catheter assembly design draws prominently upon lessons learned from a study that showed that redundant amperometric glucose sensors (with the use of averaging or voting schemes) provided more accurate glucose data than single sensing units. This published study showed that if one separates each sensing unit by at least 7 mm, there is a benefit of redundancy ({Castle, 2012 #30}. Even two sensing units showed accuracy that was substantially superior to that of a single sensing unit, especially with regard to (1) reducing the prevalence of very large sensing errors (ARD values) of over 50% and (2) detection of true hypoglycemia, which was much better with redundant sensing units than with one unit.

### Computation of True Analyte Levels:

The pairing of the inactivated GOX electrode 24 and the active GOX electrode 26 permits a linear combination where the reading (current level) of the inactivated GOX, electrode 24 is subtracted from the reading (current level) of the active GOX electrode 26 to remove the response of the GOX electrode that is due to interfering substances such as acetaminophen from the active GOX reading. Because the permeability of the inactivated GOX 36 should be the same as that of the active GOX 38, the time delay for substances permeating through the inactive GOX layer 36 should parallel through the active GOX layer 38. The two layers 24 and 26 should have equal thickness so that every substance will be equally delayed. It would be very difficult to compensate computationally for proportionately different permeation times of differing substances due to differing thicknesses of layers 36 and 38.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. A catheter designed to be indwelled in a subcutaneous, dermal or intravascular location to deliver one or more drugs, said catheter being filled with porous material.

2. The catheter of claim 1, wherein said porous material is foam.

3. The catheter of claim 1, wherein said porous material is sponge material.

4. The catheter of claim 3, wherein said sponge material is selected from a group consisting of poly vinyl alcohol, poly-l-lactic acid or expanded polytetrafluoroethylene.

5. The catheter of claim 1, wherein said porous material has a porous material density is at least 35%.

6. The catheter of claim 1, wherein said porous material has a porous material density is at least 65%.

7. A method of sensing an analyte and delivering a drug, comprising:
a. providing a sensing macrocatheter assembly, including:
i. a macrocatheter tube having an exterior surface and a proximal end;
ii. a first indicating electrode disposed on said exterior surface and including an electrochemically active layer and a layer of an active functioning enzyme of a first enzyme type on top of said electrochemically active layer;
iii. a reference electrode disposed on said exterior surface;
iv. conductive traces disposed on said exterior surface, leading from said electrodes to a location near said proximal end; and
v. an electrical and data processing system, electrically connected to said conductive traces and adapted to bias said electrodes and measure electrical signals from said electrodes, and using said signals to determine concentration of an analyte concentration and communicating said analyte concentration to a location apart from said first and second indicating electrodes;
b. indwelling said sensing macrocatheter tube in a human patient's subcutaneous, dermal or intravascular location;
c. using said indicating electrodes to sense concentration of one or more analytes, thereby forming sensed concentrations of one or more analytes; and
d. delivering one or more drugs through said macrocatheter tube in quantities responsive to said sensed concentrations of one or more analytes, but being corrected for effects of previously delivered drugs on analyte measurements.

8. The method of claim 7, in which said sensing microcatheter assembly includes a second indicating electrode, disposed on said exterior surface and including an electrochemically active layer and a layer of an inactivated enzyme of said first enzyme type on top of said electrochemically active layer.

9. The method of claim 7, in which said analyte is glucose.

10. The method of claim 7, in which said enzyme is selected from the group consisting of glucose oxidase and glucose dehydrogenase.

11. The method of claim 7, in which the delivered drug is taken from a group consisting of insulin, glucagon and pramlintide or a combination of two or more of said drugs.

12. The method of claim 7, wherein said macrocatheter tube is fabricated from a flexible material.

13. The method of claim 12, wherein said flexible material is polyimide.

14. A method of manufacturing a catheter having a sensing assembly on its exterior surface, comprising:
a. providing a catheter tube;
b. providing a flexible substrate;
c. producing a sensing assembly on a first side said flexible substrate; and
d. wrapping said flexible substrate about said catheter tube so that said first side of said flexible substrate faces out.

15. The method of claim 14, wherein said catheter tube is supported by a cylindrical support placed inside said catheter tube during said wrapping.
